(19) United States
(12) Patent Application Publication
(10) Pub. No.: US 2009/0216012 A1
(43) Pub. Date: Aug. 27, 2009

(54) USE OF CENTRIFUGAL PARTITION CHROMATOGRAPHY FOR PURIFYING GALANTHAMINE

(75) Inventors: Jean-Hugues Renault, Cauroy Les Hermonville (FR); Jean-Marc Nuzillard, Courmas (FR); Alexandre Maciuk, Malabry (FR); Monique Zeches-Hanrot, Bezannes (FR)

Correspondence Address: YOUNG & THOMPSON, 209 Madison Street, Suite 500, ALEXANDRIA, VA, 22314, US

(73) Assignees: Centre National De La Recherche Scientifique, Paris Cedex 16 (FR); Universite De Reims Champagne-Ardenne, Reims (FR)

(21) Appl. No.: 11/722,004

(22) PCT Filed: Dec. 6, 2005
(86) PCT No.: PCT/FR05/03046

§ 371 Date: Feb. 24, 2009

(30) Foreign Application Priority Data
Dec. 16, 2004 (FR) 0413425

(51) Int. Cl. C07D 491/04

(52) U.S. Cl. 540/576

(57) ABSTRACT

The invention concerns the use of centrifugal partition chromatography in displacement mode for implementing a process for purifying galanthamine or its derivatives, from a starting composition, containing at least 20% of galanthamine or its derivatives. Said method comprises a step of centrifuging a combination of at least two solvents and said starting composition, for a time sufficient for purifying the galanthamine or its derivatives, said solvents being such that they form two non-miscible phases, namely an aqueous phase and an organic phase, the aqueous phase serving as mobile phase or stationary phase, the organic phase serving respectively as stationary phase or mobile phase.

A subject of the present invention is the use of centrifugal partition chromatography for the purification of galanthamine.

Centrifugal partition chromatography (CPC) is a liquid-liquid chromatography technique without support solid (Foucault, 1994; Berthod, 2002), which requires the use of a biphasic system produced from at least two solvents and/or solutions. The columns used are of two types: either they are constituted by a hollow cylinder the wall of which is hollowed out with partition cells distributed radially and connected together by ducts with a section smaller than that of the partition cells (see French Patent Application 2.802.104 or International Application WO 2004/079363 in the name of Partus Technologies); or they are constituted by stacked discs engraved with partition cells connected together by ducts of smaller section where the seal between the discs is ensured by Teflon® joints with the same diameter as the discs (International Application WO 00/58722 in the name of Kromaton Technologies). The constant centrifugal force field generated by the rotation of the column holds one of the stationary phases in the latter, the other phase being pumped through and playing the role of mobile phase. The achievement of a separation by CPC is mainly characterized by the selection of a system of solvents suitable for a specific separation (Renault et al., 2002).

Another essential element of a separation by CPC is the choice of chromatographic development mode. Elution is a mode common to high performance liquid chromatography (HPLC) and to CPC, where the analytes are only subject to the thermodynamic laws of partition equilibria between solvents. This mode is ideally characterized by the elution of the analytes according to a Gaussian concentration profile. Another mode, called displacement, and described for the first time by Tiselius (Tiselius, 1943), is based on the competition of the analytes between themselves during chromatography. Two conditions must be fulfilled for observing this phenomenon: the non linear behaviour of each of the analytes and the mobile phase cannot mobilise alone the analytes. For this to happen a displacing agent must be introduced in solution in the mobile phase which has an interaction with the stationary phase which is stronger than all the analytes. An example is constituted by ion exchange chromatography in CPC or on solid support (Maciuk et al., 2004). The stationary phase then contains an exchanger (cationic or anionic, strong or weak) and the analytes progress in the column under the action of the displacing agent. The compounds organise themselves in the column in a succession of zones in which the concentrations are constant, the order in which the compounds emerge being fixed by their association constants with the exchanger. This train of compounds, called isotachic train, progresses in the column at a velocity function of the stoichiometric ratio between the concentration of retaining agent (compound intended to hold the analyte in the stationary phase) and the concentration of displacing agent (Maciuk et al., 2004; Intes et al., 2001). The concentration profiles of the emerging species are then rectangular and have abrupt transition zones called “schock layers”. The latter method is particularly suitable for preparative applications. A variant proper to liquid-liquid chromatography without solid support which is similar in principle to the “pH-zone refining” claimed in Counter-Current Chromatography (liquid-liquid chromatography without support solid in hydrodynamic mode) (U.S. Pat. No. 5,332,504, U.S. Pat. No. 5,354,473 and U.S. Pat. No. 5,449,461; Weisz et al., 1994) is obtained by taking advantage of the acid-base equilibria between the analytes. The compounds which can be separated by this chromatographic mode must be ionisable and the neutral and ionized forms must have sufficient differences of polarity to invert their distribution constants in the selected biphasic system (Ito et al., 1996; Ito et al., 1995; Renault et al., 1999).

As well as the advantages of CPC which are known and frequently mentioned such as the absence of irreversible adsorption of solvents, the reduced consumption of solvents, obtaining high selectivities, it appears that this technique has undeniable preparative qualities (Margraff et al., 1994; Renault et al., 1999).

Natural (−)-galanthamine (C₁₇H₂₁NO₃; M=287.2) is an amine corresponding to the following formula:

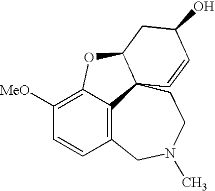

(+)-Galanthamine, which is accessible by synthesis, is the 12a(R),4a(R),3(S) enantiomer of galanthamine, and has no biological activity.

(−)-Galanthamine is a competitive inhibitor of acetylcholinesterase in the treatment of the symptoms of Alzheimer's disease. The current available treatments are only symptomatic and are all based on the cholinergic hypothesis of the disease. Among the molecules which are currently on the market, galanthamine is the only natural substance, being present in the bulbs and/or the aerial parties of certain plants of the Amaryllidaceae family. It is in particular marketed by Janssen-Cilag laboratories (Reminyl® which is a hydrobromide salt of (−)-galanthamine), but its production by Sanochemia Pharmazeutica AG (Vienna, Austria) is ensured by the chemical synthesis route, in particular according to the processes described in the International Application WO 97/45431 or in the U.S. Pat. No. 6,407,229.

Within the framework of these processes, obtaining optically pure galanthamine involves the use of chiral reagents and/or of a fractional crystallization from a salt of an optically pure organic acid (tartaric acid). It is also sometimes necessary to resort to chromatography on support solid (silica gel). Such processes are therefore long and relatively expensive to implement.

Moreover, the U.S. Pat. No. 6,573,376, U.S. Pat. No. 6,617,452 and U.S. Pat. No. 6,194,404 relate to a process for isolating galanthamine, comprising an extraction stage and a purification stage by recrystallization. The crystallization processes of the prior art, in particular perfected for an extract of Narcissus pseudonarcissus “Carlton”, requires a priori a very selective extraction in order to enrich the extract to the maximum extent with galanthamine. In addition, such processes can prove to be ineffective in the case of an extract obtained from a different plant. Finally, the extraction yield is particularly low (0.01% from the dry bulbs) starting from a drug titrating between 0.2 and 0.3%.

The purification processes used currently are processes which are onerous and difficult to implement and are expensive.

Existing purification processes are limited by the use of solid chromatographic supports, which affects the yields of isolated products, and by the use of significant quantities of solvents compared to the masses produced.

The purpose of the present invention is to provide a process for the purification of galanthamine or its derivatives, leading to obtaining compounds in a state of purity which is compatible with the requirements of the pharmaceutical industry, and with production costs which are lower than those obtained during the use of chromatography techniques on support solid.

The purpose of the present invention is to provide a process for the purification of galanthamine which is reproducible and can be easily industrialized.

The present invention relates to the use of a centrifugal partition chromatography process in displacement mode, for the implementation of a process for the purification of galanthamine or its derivatives.

The expression “displacement mode” corresponds to a CPC mode different from the elution mode. This mode is close in its principle to ion exchange.

The expression “galanthamine or its derivatives” designates galanthamine or the derivatives of galanthamine such as: sanguinine, galanthaminone (or narwedine), galanthaminone, norgalanthamine, 11-hydroxygalanthamine, lycoramine, lycorine, assoanine, etc.

The purified compound obtained, corresponding to galanthamine or to one of its derivatives, has a purity greater than approximately 99%, and advantageously greater than approximately 99.2%.

The process of the invention makes it possible to access to very high chromatographic selectivities and to purification yields exceeding 90%. In addition, the use of CPC makes it possible to reduce the quantity of solvent by a factor comprised between 5 and 10 in comparison to liquid chromatography HPLC, which is generally used for this type of purification. This solvent saving is directly attributable to the high proportion of stationary phase which is entirely accessible in the column (conventionally between 30 and 80% of the total volume of the column), which limits the dilution of the analytes. In addition, it must be emphasized that the absence of solid phase allows a high reproducibility by avoiding the problems of progressive compaction of the chromatographic support. Finally, compared to crystallization, the process is more adaptable to the different potential sources of galanthamine (different plant, synthesis, biotechnologies), and produces much better yields, on average greater than 90%.

The process of the invention allows flexibility, insofar as the purified compound can be obtained in the form of a base or a salt, according to the desired subsequent use.

Within the framework of the use of galanthamine in medicaments, galanthamine is in the hydrobromide form. Thus, when the compound is obtained in the form of a base, it is necessary to convert it to the hydrobromide and this additional stage makes it possible to improve the purity of galanthamine as it makes it possible to lose additional impurities.

The present invention relates to the use as defined above, starting from an starting composition, containing at least 20% of galanthamine or its derivatives, said process comprising a stage of centrifuging a combination of at least two solvents and said starting composition, for a sufficient time to purify the galanthamine or its derivatives,

said solvents being such that they form two non miscible phases, namely an aqueous phase and an organic phase, the aqueous phase serving as mobile phase or stationary phase, and the organic phase serving respectively as stationary phase or mobile phase.

The process of the present invention comprises a stage in which on the one hand the starting composition as defined above and on the other hand a combination of at least two solvents, as defined above, are centrifuged.

The combination of solvents always comprises at least two solvents, insofar as the process of the invention requires the presence of two phases, namely an organic phase and an aqueous phase, which is not possible in the case of the use of a single solvent.

Within the framework of the centrifugation stage, the centrifugal force field created makes it possible to hold a stationary phase in the column. Generally, the operator will wish to generate a rotation which is as fast as possible as this improves the transfer of material by increasing the exchange surface between the mobile phase and the stationary phase. The upper limit is function of the tolerance of the apparatus to pressure drop (the latter increases with rotation; the maximum is 60 bars on standard laboratory apparatus and 150 bars on the industrial device marketed by Partus). Conventionally, the applied force field is a minimum of 100 g.

The expression “for sufficient time to purify the galanthamine or its derivatives” corresponds to a duration from approximately 20 minutes to several hours.

The expression “starting composition” designates a composition containing at least the compound to be purified, namely galanthamine or one of its derivatives, i.e. containing at least one alkaloidal compound. This starting composition corresponds to the composition subjected to the purification process, before the centrifugation stage.

According to an advantageous embodiment of the present invention, the starting composition contains the compound to be purified, in the form of a salt or amine, dissolved in the stationary phase or the mobile phase, said starting composition being saturated or not by the addition of mobile phase or stationary phase respectively.

According to an advantageous embodiment, the compound to be purified is recovered in the organic phase, insofar as it will be easier to recover in this phase which is likely to be evaporated more easily than the aqueous phase.

Within the framework of the process of the invention, when the stationary phase is the aqueous phase, the mobile phase is the organic phase. Inversely, when the stationary phase is the organic phase, the mobile phase is the aqueous phase.

The present invention relates to a process for the purification of galanthamine or its derivatives from a starting composition, containing at least 20% of galanthamine or its derivatives, by centrifugal partition chromatography in displacement mode, said process comprising a stage of centrifuging

a combination of at least two solvents, and preferably at least three solvents, and

said starting composition

for a sufficient time to purify galanthamine or its derivatives,

said solvents being such that they form two non miscible phases, namely an aqueous phase and an organic phase, the aqueous phase serving as mobile phase or stationary phase, and the organic phase serving respectively as stationary phase or mobile phase.

An advantageous process according to the present invention is characterized in that the starting composition is a plant extract or biological material producing galanthamine or its derivatives, such as plant cells or a mixture of compounds obtained by organic synthesis containing in particular galanthamine and/or its derivatives.

The compounds obtained by organic synthesis also require a purification stage, which in certain cases can be a chromatography stage. In addition, if a racemic mixture is separated by the process of the invention, the racemate is obtained with other impurities removed.

Said plant extract or said biological material or said mixture is dissolved in the mobile phase or the stationary phase in order to be introduced at the column head.

The choice for the phase which will be used to dissolve the starting composition is guided by two criteria:
the solubility: it will be chosen to solubilize the starting composition in the phase where it has the best solubility for obvious reasons of productivity,
the influence of the starting composition on the equilibrium of the biphasic system: it is possible that the dissolution in a phase leads to a lower destabilization of the biphasic system, which will lead to a better injection profile and thus to better retention of the stationary phase and therefore a better separation (Marchal et al., 2003).

The compounds obtained by organic synthesis also require a purification stage, which can be, in certain cases, a chromatography stage.

If a racemic mixture is separated by the process of the invention, the racemate is obtained with other impurities removed.

A particularly advantageous process according to the present invention is characterized in that the two non-miscible liquid phases correspond to a combination of at least two solvents, namely water and a solvent which is non-miscible or partially miscible with water, thus forming an aqueous phase and an organic phase.

The expression “solvent partially miscible with water” designates a solvent which is not totally miscible with water.

Thus, for example, the butanol/water system is a biphasic system in which a significant quantity of butanol is present in the aqueous phase and in which a significant quantity of water is present in the organic phase.

The aqueous phase corresponds to a phase rich in water, i.e. essentially constituted of water. It also contains a small quantity of solvent(s) which are non-miscible or partially miscible with water.

The organic phase corresponds to a phase rich in solvent(s) which is (are) partially or non-miscible in water, i.e. essentially constituted of solvent(s) which is (are) non miscible or partially miscible with water. It also contains a small quantity of water.

The present invention relates to a process as defined above, characterized in that the two non-miscible liquid phases correspond to a mixture of at least three solvents, namely water, a solvent non-miscible or partially miscible with water and a “bridge solvent”, said bridge solvent being a solvent partially or totally soluble in water and in the solvent which is non-miscible or partially miscible with water,

said solvents being such that they form a biphasic system comprising an aqueous phase and an organic phase.

The expression “bridge solvent” is a term used within the framework of the present invention in order to designate a solvent which is both miscible in the organic phase and in the aqueous phase. It is therefore present in both phases.

The expression “solvent partially soluble in water and in the solvent which is non-miscible or partially miscible with water” designates a solvent present both in the organic phase and in the aqueous phase.

The expression “biphasic system” designates a system having two phases, which are both liquids in the case of the present invention.

According to an advantageous embodiment of the present invention, the process as defined above is characterized in that the solvent which is non-miscible or partially miscible with water is chosen from:
ethers such as methyl tert-butyl ether (MTBE) and ethyl tert-butyl ether,
ketones which are non-miscible with water such as methyl ethyl ketone (MEK) and methyl isobutyl ketone (MIBK),
aromatic hydrocarbons such as toluene,
aliphatic hydrocarbons such as hexane, heptane and the cyclohexanes,
petroleum ethers,
heavy alcohols the carbon-containing chain of which comprises at least 4 carbon atoms, such as n-butanol, 2-butanol and isobutanol,
siloxanes which are non-miscible with water such as hexamethyldisiloxane,
halogenated solvents which are non-miscible with water, such as chloroform, dichloromethane and dichloro-1,2-ethane, or
esters such as ethyl acetate and butyl acetate.

Among all these solvents, the halogenated solvents are the only ones which are heavier than water.

The families of solvents mentioned above are families of solvents which are non- or partially miscible with water. These solvents therefore generate a phase which is less polar than the aqueous phase. This organic phase thus has physico-chemical properties such as a dielectric constant and a dipole moment lower than water (covering the notion of apolar solvent) which is likely to allow preferential dissolution of alkaloids (basic form).

The present invention relates to a process as defined above, characterized in that the “bridge” solvent is chosen from: light alcohols the carbon-containing chain of which comprises less than 4 carbon atoms such as methanol, ethanol, propanols, acetonitrile, acetone, tetrahydrofuran, dimethylsulphoxide and dimethylformamide.

These solvents are “good solvents” of the alkaloids (salt form and basic form) facilitating general solubilization and also the transfer of material between the two phases.

The present invention relates to a process as defined above, characterized in that the mobile phase contains a displacing agent which is an acid or a base.

In displacement mode in general, the solvents constituting the mobile phase are not eluents on their own. They are incapable of mobilizing the compounds to be purified which are present in the starting composition.

An additional species, introduced into the system by the mobile phase, displaces the compounds to be purified which are present in the starting composition from their fixation on the stationary phase: this is the displacing agent, or developer. Like the retaining agent, it has the same type of interaction with the stationary phase as the compounds to be purified, but with an association constant with the exchanger which is greater than the latter. It is the displacing agent which is at the origin of the motive force of the chromatography.

In “pH-zone-refining” mode, the displacing agent is a species present in the mobile phase, which forces the compound to be purified to pass into the mobile phase, by causing a change in its ionization state by acidic/basic reactions. In the case of the separation of alkaloids, if the mobile phase is organic, the displacing agent must be a liposoluble strong base capable of neutralizing the compounds to be purified and of reducing their K_{D} (distribution constant). If the mobile phase is aqueous, the displacing agent must be a hydrosoluble strong acid capable of protonating the compounds to be purified and of increasing their K_{D}. The displacing agent is necessarily added to the mobile phase, it cannot be injected with the sample. It is its concentration that determines the velocity of the compounds to be purified in the column.

According to the present invention, the displacing agent is an acid/basic compound present in the aqueous or organic mobile phase.

An advantageous process according to the present invention is a process as defined above, characterized in that the stationary phase corresponds to the aqueous phase and the mobile phase corresponds respectively to the organic phase, and in that the displacing agent contained in the organic mobile phase is a base.

According to this embodiment, the purified alkaloidal compound, namely galanthamine or one of its derivatives, is eluted in the mobile phase, i.e. in the organic phase; it is therefore in its basic form, i.e. in an amine form. Thus, the alkaloidal compound to be purified is initially in its acidic form, namely in the form of an ammonium salt.

An advantageous process according to the present invention is a process as defined above, characterized in that the stationary phase corresponds to the organic phase and the mobile phase corresponds respectively to the aqueous phase, and in that the displacing agent contained in the aqueous mobile phase is an acid.

According to this embodiment, the purified compound, namely galanthamine or one of its derivatives, is eluted in the mobile phase, i.e. in the aqueous phase; it is therefore in its acidic form, i.e. in its ammonium salt form. Thus, the initial form of compound to be purified is its basic form, namely the amine form.

A preferred process according to the invention is characterized in that a retaining agent is:
either added in the starting composition or in the stationary phase,
or is an element of the starting composition,

said retaining agent being an acid or a base.

The expression “retaining agent” designates a compound subject to the purification process having the greatest difference of pKa with the displacing agent as defined above.

The name of retaining agent is given to a species of the same acid/basic character as the compound to be purified, but showing a greater difference of pKa with the displacing agent than all the species present. The retaining agent holds the compound to be purified in the stationary phase. The retaining agent can be present in the stationary phase, or can be injected with the sample (a particular case is then to consider that the compound of the starting composition having the greatest difference of pKa with the displacing agent plays this role). In the case of a basic compound to be purified, if the stationary phase is aqueous, the retaining agent must be a hydrosoluble strong acid, capable of ionising the compound to be purified in order to promote its partition in the stationary phase (by increasing its distribution constant K_{D} which must be >>1). If the stationary phase is organic, the retaining agent must be a liposoluble strong base capable of neutralising the compound to be purified and increasing its constant of distribution which must be <<1).

The process of the invention therefore allows the purity/purification time relationship to be controlled, insofar as the quantities of displacing agent and retaining agent—governing the speed of progression of the isotachic train of compounds—can be modulated in order to obtain a compound emergence time just sufficient to obtain the desired purity.

According to a preferred embodiment, the process of the invention is characterized in that a retaining agent, which is an acid, is added in the starting composition or in the stationary phase.

According to this embodiment, the organic mobile phase contains a basic displacing agent and the stationary phase is the aqueous phase, into which an acidic retaining agent is added having an acidity constant higher than those of the protonated forms of the alkaloidal compounds to be purified.

According to a preferred embodiment, the process of the invention is characterized in that a retaining agent, which is a base, is added in the starting composition or in the stationary phase.

According to this embodiment, the aqueous mobile phase contains an acidic displacing agent and the stationary phase is the organic phase, into which a basic retaining agent is added having an acidity constant lower than those of the alkaloidal compounds to be purified.

According to an advantageous embodiment, the process of the present invention is characterized in that the starting composition contains the alkaloids in the form of ammonium salts dissolved in the aqueous stationary phase, the starting composition then being saturated or not by an addition of organic mobile phase without displacing agent.

One of the benefits of CPC is the ability to inject in the mobile phase or in the stationary phase. Three aspects are to be considered:
the solubility of the starting composition (as much as possible should be dissolved in order to be productive etc.),
the influence of the starting composition on the biphasic character of the solvent system, and in this case one of the two phases can have a better affinity than the other (Marchal et al., 2003),
the final quality of the separation.

It has been shown that one of the conditions for a successful separation was the control of the saturated character in conjugated phase (organic phase or aqueous phase) of the phase in which the sample was dissolved (aqueous phase or organic phase respectively) (Marchal et al., 2003). This is because a quantity of “virgin” conjugated phase (organic phase or aqueous phase) can be added in order to resaturate the injection volume.

According to another advantageous embodiment, the process of the present invention is characterized in that the starting composition contains the alkaloids in the form of neutral bases dissolved in the aqueous mobile phase, the starting composition then being saturated or not by the addition of organic stationary phase without retaining agent.

According to another advantageous embodiment, the process of the present invention is characterized in that the starting composition contains the alkaloids in the form of neutral bases dissolved in the organic stationary phase, the starting composition then being saturated or not by the addition of aqueous mobile phase without displacing agent.

According to another advantageous embodiment, the process of the present invention is characterized in that the starting composition contains the alkaloids in the form of ammonium salts dissolved in the organic mobile phase, the starting composition then being saturated or not by the addition of stationary aqueous phase without retaining agent.

The present invention also relates to a process as defined above, comprising the following stages:
the injection of the stationary phase into a centrifugal partition chromatography column, said stationary phase containing a retaining agent as defined above, in order to obtain a centrifugal partition chromatography column filled with stationary phase,
the injection of the starting composition as defined above into the centrifugal partition chromatography column filled with stationary phase, in order to obtain a centrifugal partition chromatography column loaded with said stationary phase and said starting composition, and
the introduction by pumping into the column as obtained in the previous stage, of the mobile phase in which a displacing agent is added as defined above, in order to elute galanthamine or its derivatives.

The expression “centrifugal partition chromatography column filled with stationary phase” designates a column, corresponding to a succession of parallelepipedic or cylindrical cavities, connected together by channels. At the start, the column is empty. Each separation by CPC therefore begins by completely filling the column, generally by applying a slow rotation (100 to 300 rpm).

Once the column is filled with stationary phase, the working rotation is applied (conventionally between 100 and 500 g). The sample is injected via an injection loop or via the pomp, by being pushed by the mobile phase. The mobile phase will displace a volume of stationary phase in each partition cell which depends on the system of solvents, the rotation, the flow rate, until reaching a hydrodynamic equilibrium. This equilibrium is characterized by the ratio volume of stationary phase/total volume of the column (noted Sf and called retention). Concretely, at the start of the experiment, output of the stationary phase is observed, until reaching equilibrium, detected by the appearance (the output) of the mobile phase. In this state of equilibrium, for each volume of mobile phase pumped, the same volume of mobile phase outputs the column. A CPC chromatographic system is therefore characterized in part by the rate of retention of stationary phase in the column. Conventionally, separations are possible with retentions comprised between 30 and 85%.

The process of the invention as defined above is characterized in that the displacing agent is chosen from:
mineral acids such as HCl or H₂SO₄,
organic acids such as methanesulphonic acid, trifluoroacetic acid, acetic acid, tartaric acid or citric acid,
mineral bases such as ammonia or soda, or
organic bases such as triethylamine.

When the displacing agent is an acid, it is more acidic than the conjugated acids of the compounds present in the starting composition, and when the displacing agent is a base, it is more basic than the compounds present in the starting composition.

The mineral acids are the products most usually used as displacing agents. In the particular case of galanthamine, HBr can be useful insofar as the pharmaceutical form is galanthamine hydrobromide. Tartaric acid is useful as it is then possible to extract the alkaloids in the form of tartrates which can be pharmaceutical salts.

Among the bases, the preferred displacing agents are the organic bases as in this case, the basic displacing agent must show a correct solubility in the organic phase.

The process of the invention as defined above is characterized in that the retaining agent is chosen from:
mineral bases such as ammonia or soda, or
organic bases such as triethylamine.
mineral acids such as HCl or H₂SO₄,
organic acids such as methanesulphonic acid, trifluoroacetic acid, acetic acid, tartaric acid or citric acid.

When the retaining agent is an acid, it is more acidic than the conjugated acids of the compounds present in the starting composition and, when the retaining agent is a base, it is more basic than the compounds present in the starting composition.

According to an advantageous embodiment of the invention, the process of the invention comprises the use of a combination of the following solvents:
toluene, heptane, acetone and water, or
methyl tert-butyl ether, acetonitrile and water, or
methyl tert-butyl ether, acetone and water, or
methyl isobutyl ketone, acetone and water.

The present invention also relates to a process as defined above, comprising the use of the following combination of solvents: toluene, heptane, acetone and water, in volume proportions such that:
the volume percentage of toluene is greater than that of heptane,
the volume percentage of acetonitrile does not exceed 50%, and
the mixture of these solvents is biphasic,

and in particular in the volume proportions 24:8:10:34.

The present invention also relates to a process as defined above, comprising the use of the following combination of solvents: methyl tert-butyl ether, acetonitrile and water, in volume proportions such that:
the volume percentage of the acetonitrile does not exceed 45%, and
the mixture of these solvents is biphasic,

and in particular in the volume proportions 4:1:5.

According to an advantageous embodiment, the process of the invention is characterized in that the starting composition is an extract of aerial parts or bulbs of Amaryllidaceae, in particular of the genus Leucojum, Narcissus or Galanthus, and is preferably an extract of leaves of Leucojum aestivum or an extract of bulbs of Narcissus carlton.

According to an advantageous embodiment, the process of the invention is characterized in that the starting composition is an extract of leaves of Leucojum aestivum and in that the combination of solvents is as follows: toluene, heptane, acetone and water, in the volume proportions 24:8:10:34.

According to an advantageous embodiment, the process of the invention is characterized in that the starting composition is an extract of bulbs of Narcissus carlton and in that the combination of solvents is as follows: methyl tert-butyl ether, acetonitrile and water, in the volume proportions 4:1:5.

The process of the present invention can be carried out in ascending mode or descending mode. The ascending mode corresponds to the case where the mobile phase is lighter than the stationary phase, while the descending mode corresponds to the case where the mobile phase is heavier than the stationary phase.

Within the framework of the present invention, the organic phase is always lighter than the aqueous phase, except when the organic phase comprises chlorinated solvents such as chloroform, the dichloromethane and dichloro-1,2-ethane.

The present invention also relates to a process as defined above, characterized in that it comprises the following stages:
the injection of the aqueous stationary phase into a centrifugal partition chromatography column, said stationary phase containing an acidic retaining agent as defined above, in order to obtain a centrifugal partition chromatography column filled with acidified stationary phase,
the injection of the starting composition, in which galanthamine or its derivatives are in the form of salts, into the centrifugal partition chromatography column filled with acidified stationary phase, in order to obtain a centrifugal partition chromatography column loaded with said acidified stationary phase and said starting composition, and
the introduction by pumping of the organic mobile phase through the column as obtained in the previous stage, in which a basic displacing agent is added, in order to elute galanthamine or its derivatives in basic form.

Within the framework of the implementation of the above-mentioned process, just before the injection of the organic mobile phase, the column contains at the head, in the first partition cells, the sample (analyte in the form of salt) then the aqueous stationary phase in which the acidic retaining agent has been added. Then, the organic mobile phase containing the basic displacing agent is injected. The basic displacing agent then takes the proton of the analyte salt which is the least basic of the starting composition and entrains this analyte (amine form) in the organic mobile phase, which advances in the column because of the pumping. Thus, the analyte (amine form) displaced is protonated by the acidic retaining agent during contact with the stationary phase and is then found in its salt form in the stationary phase.

The process of the invention therefore corresponds to a sequence of different acidic/basic reactions which entrain the displacement of the compound to be purified between the stationary phase and the aqueous phase.

According to an advantageous embodiment, the process as defined above, in particular in the paragraph above, is characterized in that the acidic retaining agent is added in the stationary phase.

The present invention also relates to a process as defined above, characterized in that it comprises the following stages:
the injection of the organic stationary phase into a centrifugal partition chromatography column, said stationary phase containing a basic retaining agent as defined above, in order to obtain a centrifugal partition chromatography column filled with an alkalinized stationary phase,
the injection of the starting composition, in which galanthamine or its derivatives are in basic form, into the centrifugal partition chromatography column filled with alkalinized stationary phase, in order to obtain a centrifugal partition chromatography column loaded with said alkalinized stationary phase and said starting composition, and
the introduction by pumping of the aqueous mobile phase through the column as obtained in the previous stage, in which an acidic displacing agent is added, in order to elute galanthamine or its derivatives in the form of salts.

According to an advantageous embodiment, the process as defined above, in particular in the paragraph above, is characterized in that the basic retaining agent is added in the stationary phase.

### EXPERIMENTAL PART

### Example 1

### Leucojum aestivum Ascending Mode

1—Preparation of the Extract:

1.660 kg of crushed leaves of Leucojum aestivum (2 mm screen) are moistened with 1 litre of 10% ammonia in water. The mixture is placed in a percolator then is macerated in 30 l of ethyl acetate for 18 h. Then, the ethyl acetate is leached to exhaustion. The extractive solution is extracted with 3×3 l then 3×2 l of 3% sulphuric water. The aqueous phase is alkalinized to pH approximately 10 by 20% ammonia in water. This aqueous solution is extracted with 2×3 l then 1×2 l of chloroform. The chloroformic solution is washed with water until a pH of approximately 7 is obtained, dried over sodium sulphate then evaporated to dryness under reduced pressure in order to obtain 3.639 g of extract of total alkaloids, i.e. a yield of 2.192 g/kg of dry leaves.

2—Separation by Centrifugal Partition Chromatography, in Displacement Mode:

a—Apparatus.

The device used is a FCPC Centrifugal Partition Chromatograph Kromaton® with a capacity of 200 ml (Kromaton Technologies, Angers, France).

b—Biphasic System of Solvents.

The system used corresponds to a toluene/heptane/acetone/water mixture 24:8:10:34 (v/v). The solvents are stirred in a separating funnel, then separated. The aqueous phase is acidified by methanesulphonic acid (retaining agent) at a concentration of 10 mM. The organic phase is alkalinized by triethylamine (displacing agent) at a concentration of 8 mM. The aqueous phase is chosen as the stationary phase, the organic phase as the mobile phase.

c—Implementation of the Separation

2.772 g of extract obtained according to the method described above is dissolved in 20 ml of methanol. The solution is acidified (test with pH paper) by methanesulphonic acid in order to ionize the alkaloidal species. The methanol is then evaporated off under reduced pressure, then the syrupy residue is dissolved in 20 ml of acidified aqueous stationary phase and 1 ml of neutral organic mobile phase.

The column is filled with stationary phase (300 rpm, ascending mode, 20 ml/min), then the rotation is fixed at 1700 rpm. The injection volume is then introduced into the column via an injection loop, pushed by the mobile phase at 8 ml/min in ascending mode. The elution is continued for 2 hours, the retention percentage of the stationary phase being 70% and the pressure drop being 68 bars. No leakage was observed. The effluent is fractionated using an automatic fraction collector (SuperFrac Pharmacia). Detection is carried out under UV (254 nm), and by an in-line check of the pH. Galanthamine is eluted between the fractions 62 and 100. After evaporation of the solvents under reduced pressure, 1.219 g of galanthamine base is obtained (44% of the extract). The purity before recrystallization was estimated to exceed 99% by thin layer chromatography (TLC), HPLC then by NMR ¹H and ¹³C (Bruker DRX 500 MHz).

Other minority compounds have also been isolated. These are: narwedine, norgalanthamine and ungiminorine.

### Example 2

### Leucojum aestivum Descending Mode

a—Biphasic System of Solvents.

The system used corresponds to a toluene/heptane/acetone/water mixture 24:8:10:34 (v/v). The solvents are stirred in a separating funnel, then separated. The aqueous phase is acidified by methanesulphonic acid (displacing agent) at a concentration of 8 mM. The organic phase is alkalinized by triethylamine (retaining agent) at a concentration of 10 mM. The aqueous phase is chosen as the mobile phase, the organic phase as the stationary phase.

b—Separation by CPC

2.6 g of extract obtained according to the method described above is dissolved in 20 ml of alkalinized organic stationary phase and 1 ml of neutral aqueous mobile phase.

The column is filled with organic stationary phase (300 rpm, ascending mode, 20 ml/min), then the rotation is fixed at 1700 rpm. The injection volume is then introduced into the column via an injection loop then pushed by the mobile phase at 8 ml/min in descending mode. The elution is continued for 2 hours, the retention percentage of the stationary phase being 65% and the pressure drop being 67 bars. No leakage of stationary phase is observed. The effluent is fractionated using an automatic fraction collector (SuperFrac Pharmacia). Detection is carried out under UV (254 nm), and by an in-line check of the pH. Analysis of the fractions by TLC allows those containing galanthamine to be detected. They are combined, then evaporated under reduced pressure. 1.142 g of galanthamine base is obtained (44% of the extract). The purity before recrystallization was estimated to exceed 99% by TLC, HPLC then by NMR ¹H and ¹³C (Bruker DRX 500 MHz).

### Example 3

### Narcissus Carlton Ascending Mode

a—Biphasic System of Solvents.

The system used corresponds to a methyl tert-butyl ether/acetonitrile/water mixture 4:1:5 (v/v). The solvents are stirred in a separating funnel, then separated. The aqueous phase is acidified by methanesulphonic acid (retaining agent) at a concentration of 20 mM. The organic phase is alkalinized by triethylamine (displacing agent) at a concentration of 16 mM. The aqueous phase is chosen as stationary phase, the organic phase as mobile phase.

b—Separation by CPC

5 g of alkaloidal extract of Narcissus Carlton bulbs obtained according to the method described above is dissolved in 20 ml of methanol. The solution is acidified to pH approximately 3 (test with pH paper) by methanesulphonic acid in order to ionize the alkaloidal species. The methanol is then evaporated off under reduced pressure, then the syrupy residue is dissolved in 20 ml of acidified aqueous stationary phase and 1 ml of neutral organic mobile phase.

The column is filled with stationary phase (300 rpm, ascending mode, 20 ml/min), then the rotation is fixed at 1200 rpm. The injection volume is then introduced into the column via an injection loop, pushed by the mobile phase at 8 ml/min in ascending mode. The elution is continued for 3 hours, the retention percentage of the stationary phase being of 66% and the pressure drop of 42 bars. No leakage of stationary phase is observed. The effluent is fractionated using an automatic fraction collector (SuperFrac Pharmacia). Detection is carried out under UV (254 nm), and by an in-line check of the pH. Galanthamine is eluted between 80 and 135 minutes. After evaporation of the solvents under reduced pressure, 2.031 g of galanthamine base (amine form) is obtained. The purity before recrystallization was estimated to exceed 99% by TLC, HPLC then by NMR ¹H and ¹³C (Bruker DRX 500 MHz).

### Example 4

### Narcissus Carlton Descending Mode

a—Biphasic System of Solvents.

The system used corresponds to a methyl tert-butyl ether/acetonitrile/water mixture 4:1:5 (v/v). The solvents are stirred in a separating funnel, then separated. The aqueous phase is acidified by methanesulphonic acid (displacing agent) at a concentration of 16 mM. The organic phase is alkalinized by triethylamine (retaining agent) at a concentration of 20 mM. The aqueous phase is chosen as the mobile phase, the organic phase as the stationary phase.

b—Separation by CPC

5 g of alkaloidal extract of the bulbs of Narcissus Carlton obtained according to the method described above is dissolved in 20 ml of alkalinized organic stationary phase and 1 ml of neutral aqueous mobile phase.

The column is filled with stationary phase (300 rpm, ascending mode, 20 ml/min), then the rotation is fixed at 1200 rpm. The injection volume is then introduced into the column via an injection loop, pushed by the mobile phase at 8 ml/min (descending mode). The elution is continued for 2 hours, the retention percentage of the stationary phase being 61% and the pressure drop 47 bars. No leakage of stationary phase is observed. The effluent is fractionated using an automatic fraction collector (SuperFrac Pharmacia). Detection is carried out under UV (254 nm), and by an in-line check of the pH. Galanthamine is eluted between 71 and 127 minutes. After evaporation of the solvents under reduced pressure, 2.650 g of galanthamine in the form of methanesulphonate is obtained. The purity before recrystallization was estimated to exceed 99% by TLC, HPLC then by NMR ¹H and ¹³C (Bruker DRX 500 MHz).

### Example 5

### Narcissus Carlton (without Retaining Agent) Ascending Mode

a—Biphasic System of Solvents:

The system used corresponds to a methyl tert-butyl ether/acetonitrile/water mixture 4:1:5 (v/v). The solvents are stirred in a separating funnel, then separated. The organic phase is alkalinized by triethylamine at a concentration of 16 mM. The organic phase remains neutral. The aqueous phase is chosen as the stationary phase, the organic phase as the mobile phase.

b—Separation by CPC

5 g of alkaloidal extract of bulbs of Narcissus Carlton obtained according to the method described above is dissolved in 20 ml of methanol. The solution is acidified to pH approximately 3 (test with pH paper) by methanesulphonic acid in order to ionize the alkaloidal species. The methanol is then evaporated off under reduced pressure, then the syrupy residue is dissolved in 20 ml of acidified aqueous stationary phase and 1 ml of neutral organic mobile phase.

The column is filled with stationary phase (300 rpm, ascending mode, 20 ml/min), then the rotation is fixed at 1200 rpm. The injection volume is then introduced into the column via an injection loop, pushed by the mobile phase at 8 ml/min. The elution is continued for 110 minutes, the retention percentage of the stationary phase being 67% and the pressure drop being 41 bars. No leakage of stationary phase is observed. The effluent is fractionated using an automatic fraction collector (SuperFrac Pharmacia). Detection is carried out under UV (254 nm), and by an in-line check of the pH. Galanthamine is eluted between 45 and 100 minutes. After evaporation of the solvents under reduced pressure, 1.660 g of galanthamine base is obtained. The purity before recrystallization was estimated to exceed 99% by TLC, HPLC then by NMR ¹H and ¹³C (Bruker DRX 500 MHz).

### REFERENCES

Berthod, A. (2002) Ed. Countercurrent chromatography—The support-free liquid phase, Elsevier Science B.V.,
Foucault, A. P. (1994) Ed. Centrifugal Partition Chromatography—Theory and Practice, Marcel Dekker, New York,
Intes, O., Renault, J. H., Sinquin, C., Zèches-Hanrot, M., Nuzillard, J. M. (2001) J. Chromatography A., 918, 47-57,
Ito, Y. (1995) Modern Countercurrent Chromatography,
Ito, Y., Ma, Y. (1996) J. Chromatography A., 753, 1-36,
Maciuk, A, Renault, J. H., Margraff, R., Trébuchet, P., Zèches-Hanrot, M., Nuzillard, J. M. (2004) Anal. Chem., 76, 6179-6186,
Marchal L., Intes, O., Foucault, A., Legrand, J., Nuzillard, J. M., Renault, J. H. (2003) J. Chromatography A., 1005, 51-62,
Margraff, R. (1994) Ed. Centrifugal Partition Chromatography, Chromatographic Sciences Series, vol. 68, Marcel Dekker, New York, ch. 12, p. 331-353,
Renault, J. H., Nuzillard, J. M., Le Crouéour, G., Thépenier, P., Zèches-Hanrot, M., Le Men-Oliver, L. (1999) J. Chromatography A., 849, 421-431,
Renault, J. H., Nuzillard, J. M., Intes, O., Maciuk, A. (2002) Countercurrent chromatography, Comprehensive Analytical Chemistry, vol. 38, Elsevier, Amsterdam, ch. 3, p. 49-83,
Tiselius, A. (1943) Arkiv för Kemi och Geologo, 16A, 1-11,
Weisz, A., Scher, A., Shinomiya, K., Fales, H. M., Ito, Y. (1994) J. Am. Chem. Soc., 116, 704-708.

1-27. (canceled)

28. A method for the implementation of a process for the purification of galanthamine or its derivatives by means of centrifugal partition chromatography in displacement mode.

29. The method according to claim 28, using a starting composition, containing at least 20% of galanthamine or its derivatives, said process comprising a stage of centrifuging a combination of at least two solvents and said starting composition, for a sufficient time to purify galanthamine or its derivatives,
said solvents being such that they form two non-miscible phases, namely an aqueous phase and an organic phase, the aqueous phase serving as the mobile phase or the stationary phase, and the organic phase serving respectively as the stationary phase or the mobile phase.

30. A process for the purification of galanthamine or its derivatives from a starting composition, containing at least 20% of galanthamine or its derivatives, by centrifugal partition chromatography in displacement mode, said process comprising a stage of centrifuging
a combination of at least two solvents, and
said starting composition
for a sufficient time to purify galanthamine or its derivatives,
said solvents forming two non-miscible phases, namely an aqueous phase and an organic phase, the aqueous phase serving as the mobile phase or the stationary phase, and the organic phase serving respectively as the stationary phase or the mobile phase.

31. The process according to claim 30, wherein the starting composition is a plant extract or biological material producing galanthamine or its derivatives, or a mixture of compounds obtained by organic synthesis containing galanthamine and/or its derivatives, said plant extract or said biological material or said mixture being dissolved in the mobile phase or the stationary phase.

32. The process according to claim 30 wherein the two non-miscible liquid phases correspond to a combination of at least two solvents, namely water and a solvent which is non-miscible or partially miscible with water, thus forming an aqueous phase and an organic phase.

33. The process according to claim 30, wherein the two non-miscible liquid phases correspond to a mixture of at least three solvents, namely water, a solvent which is non-miscible or partially miscible with water and a “bridge solvent”, said bridge solvent being a solvent partially or totally soluble in water and in the solvent which is non-miscible or partially miscible with water,
said solvents forming a biphasic system comprising an aqueous phase and an organic phase.

34. The process according to claim 32 wherein the solvent which is non-miscible or partially miscible with water is chosen from:
ethers selected in the group consisting of methyl tert-butyl ether (MTBE) and ethyl tert-butyl ether,
ketones which are non-miscible with water selected in the group consisting of methyl ethyl ketone (MEK) and the methyl isobutyl ketone (MIBK),
aromatic hydrocarbons,
aliphatic hydrocarbons selected in the group consisting of hexane, heptane and the cyclohexanes,
petroleum ethers,
heavy alcohols the carbon-containing chain of which comprises at least 4 carbon atoms, selected in the group consisting of n-butanol, 2-butanol and isobutanol,
siloxanes which are non-miscible with water,
halogenated solvents which are non-miscible with water, selected in the group consisting of chloroform, dichloromethane and dichloro-1,2-ethane, or
esters selected in the group consisting of ethyl acetate and butyl acetate.

35. The process according to claim 33, wherein the “bridge” solvent is chosen from: light alcohols the carbon-containing chain of which comprises less than 4 carbon atoms.

36. The process according to claim 33, wherein the “bridge” solvent is selected in the group consisting of methanol, ethanol, propanols, acetonitrile, acetone, tetrahydrofuran, dimethylsulphoxide and dimethylformamide.

37. The process according to claim 30 wherein the mobile phase contains a displacing agent which is an acid or a base.

38. The process according to claim 30, wherein the stationary phase corresponds to the aqueous phase and the mobile phase corresponds respectively to the organic phase, and wherein the organic mobile phase contains a displacing agent which is a base.

39. The process according to claim 30, wherein the stationary phase corresponds to the organic phase and the mobile phase corresponds respectively to the aqueous phase, and wherein the aqueous mobile phase contains a displacing agent which is an acid.

40. The process according to claim 30, wherein a retaining agent is:
either added in the starting composition or in the stationary phase,
or is an element of the starting composition,
said retaining agent being an acid or a base.

41. The process according to claim 38, wherein a retaining agent, which is an acid, is added in the starting composition or in the stationary phase.

42. The process according to claim 39, wherein a retaining agent, which is a base, is added in the starting composition or in the stationary phase.

43. The process according to claim 30, comprising the following stages:
the injection of the stationary phase into a centrifugal partition chromatography column, said stationary phase containing a retaining agent, which is an acid or a base, in order to obtain a centrifugal partition chromatography column filled with stationary phase,
the injection of the starting composition into the centrifugal partition chromatography column filled with stationary phase, in order to obtain a centrifugal partition chromatography column loaded with said stationary phase and said starting composition, and
the introduction by pumping into the column as obtained in the previous stage, of the mobile phase in which a displacing agent is added, which is either a base or an acid, in order to elute galanthamine or its derivatives.

44. The process according to claim 43, wherein the stationary phase corresponds to the aqueous phase and the mobile phase corresponds respectively to the organic phase, and wherein the organic mobile phase contains a displacing agent which is a base.

45. The process according to claim 43, wherein the stationary phase corresponds to the organic phase and the mobile phase corresponds respectively to the aqueous phase, and wherein the aqueous mobile phase contains a displacing agent which is an acid.

46. The process according to claim 37, wherein the displacing agent is chosen from:
mineral acids selected in the group consisting of HCl or H₂SO₄,
organic acids selected in the group consisting of methanesulphonic acid, trifluoroacetic acid, acetic acid, tartaric acid or citric acid,
mineral bases selected in the group consisting of ammonia or soda, or
organic bases.

47. The process according to claim 40, wherein the retaining agent is chosen from:
mineral bases selected in the group consisting of ammonia or soda, or
organic bases.
mineral acids selected in the group consisting of HCl or H₂SO₄,
organic acids selected in the group consisting of methanesulphonic acid, trifluoroacetic acid, acetic acid, tartaric acid or citric acid,

48. The process according to claim 30, comprising the use of a combination of the following solvents:
toluene, heptane, acetone and water, or
methyl tert-butyl ether, acetonitrile and water, or
methyl tert-butyl ether, acetone and water, or
methyl isobutyl ketone, acetone and water.

49. The process according to claim 48, comprising the use of the following combination of solvents: toluene, heptane, acetone and water, where:
the volume percentage of the toluene exceeds that of the heptane,
the volume percentage of the acetonitrile does not exceed 50%, and
the mixture of these solvents is biphasic.

50. The process according to claim 21, wherein the volume proportions of toluene, heptane, acetone and water are 24:8:10:34.

51. The process according to claim 48, comprising the use of the following combination of solvents: methyl tert-butyl ether, acetonitrile and water, where:
the volume percentage of the acetonitrile does not exceed 45%, and
the mixture of these solvents is biphasic.

52. The process according to claim 48, wherein the volume proportions of methyl tert-butyl ether, acetonitrile and water are 4:1:5.

53. The process according to claim 30, wherein the starting composition is an extract of aerial parts or bulbs of Amaryllidaceae, of the genus Leucojum, Narcissus or Galanthus.

54. The process according to claim 53, wherein the starting composition is an extract of leaves of Leucojum aestivum or an extract of bulbs of Narcissus carlton.

55. The process according to claim 30 wherein the starting composition is an extract of leaves of Leucojum aestivum and wherein the combination of solvents is as follows: toluene, heptane, acetone and water, in the volume proportions 24:8:10:34.

56. The process according to claim 30, wherein the starting composition is an extract of bulbs of Narcissus carlton and wherein the combination of solvents is as follows: methyl tert-butyl ether, acetonitrile and water, in the volume proportions 4:1:5.

57. The process according to claim 30, wherein the stationary phase corresponds to the aqueous phase and the mobile phase corresponds respectively to the organic phase, wherein the organic mobile phase contains a displacing agent which is a base, and wherein a retaining agent, which is an acid, is added in the starting composition or in the stationary phase, said process comprising the following stages:
the injection of the aqueous stationary phase into a centrifugal partition chromatography column, said stationary phase containing an acidic retaining agent, in order to obtain a centrifugal partition chromatography column filled with acidified stationary phase,
the injection of the starting composition, in which galanthamine or its derivatives are in the form of salts, into the centrifugal partition chromatography column filled with acidified stationary phase, in order to obtain a centrifugal partition chromatography column loaded with said acidified stationary phase and said starting composition, and
the introduction by pumping of the organic mobile phase through the column as obtained in the previous stage, in which a basic displacing agent is added, in order to elute galanthamine or its derivatives in basic form.

58. The process according to claim 57, wherein the acidic retaining agent is added in the stationary phase.

59. The process according to claim 30 wherein the stationary phase corresponds to the organic phase and the mobile phase corresponds respectively to the aqueous phase, wherein the aqueous mobile phase contains a displacing agent which is an acid and wherein a retaining agent, which is a base, is added in the starting composition or in the stationary phase, said process comprising the following stages:
the injection of the organic stationary phase into a centrifugal partition chromatography column, said stationary phase containing a basic retaining agent, in order to obtain a centrifugal partition chromatography column filled with alkalinized stationary phase,
the injection of the starting composition, in which galanthamine or its derivatives are in basic form, into the centrifugal partition chromatography column filled with alkalinized stationary phase, in order to obtain a centrifugal partition chromatography column loaded with said alkalinized stationary phase and said starting composition, and
the introduction by pumping of the aqueous mobile phase through the column as obtained in the previous stage, in which an acidic displacing agent is added, in order to elute galanthamine or its derivatives in the form of salts.

60. The process according to claim 59, wherein the basic retaining agent is added in the stationary phase.
